(19) United States
(12) Patent Application Publication
(10) Pub. No.: US 2008/0249628 A1
(43) Pub. Date: Oct. 9, 2008

(54) Multi-component interbody device

(76) Inventors: Moti Altarac, Irvine, CA (US); Tyler Jay Haskins, Oceanside, CA (US); Robert C. Gutierrez, Huntington Beach, CA (US)

Correspondence Address: RIMAS LUKAS;VERTIFLEX, INC., 1351 CALLE AVANZADO, SAN CLEMENTE, CA, 92673, US

(21) Appl. No.: 12/082,303

(22) Filed: Apr. 9, 2008

Related U.S. Application Data
(60) Provisional application No. 60/922,639, filed on Apr. 9, 2007.

(51) Int. Cl. A61F 2/44

(52) U.S. Cl. 623/17.16; 623/17.11

(57) ABSTRACT

A dynamic device for implantation within a disc space between two adjacent vertebral bodies is disclosed. The device comprises two or more bodies interconnected via at least one connecting means such that at least one body is permitted relative motion with respect to the at least one other body. The device is configured to assume various configurations including a first configuration suitable for minimally invasive insertion and a second configuration suitable for residence within the disc space. Associated variations and insertion instruments are also disclosed.

### CROSS-REFERENCE TO RELATED APPLICATIONS

This application claims benefit of U.S. Provisional Patent Application Ser. No. 60/922,639 entitled “Multi-component interbody device” filed on Apr. 9, 2007, hereby incorporated by reference in its entirety.

### FIELD

The present invention generally relates to medical devices and methods. More particularly, but not exclusively, the present invention relates to inter-vertebral body spinal implants.

### BACKGROUND

The spinal column is formed from a number of vertebrae that are separated from one another by cartilaginous intervertebral discs. These discs form a cushion between adjacent vertebrae, resisting compression along the support axis of the spinal column, but permitting limited movement between the vertebrae to provide flexibility. Advancing age, disease, or other degenerative disorders as well as injury, can lead to degenerative changes in the bones, discs, joints and ligaments of the spine and may cause one or more intervertebral discs to deteriorate or become dislocated in some way, producing pain and instability.

A number of methods and associated devices have been suggested for the replacement of damaged intervertebral discs, and various methods of vertebral stabilization have been developed. For example, one common approach is to permanently stabilize or “fuse” adjacent vertebrae to maintain the proper intervertebral spacing and eliminate the relative motion between the fused vertebrae. This surgery involves removal of the affected disc and fusion of the adjacent vertebrae. In a spinal fusion procedure, a surgeon implants a spacer containing bone graft material between the vertebrae to encourage bone growth across the intervertebral space with the objective of fusing the adjacent vertebra into one bone mass. Under certain circumstances, alleviation of the problems can be provided and intervertebral spacing restored by utilizing fusion devices.

The use of such implants in treating spinal instability and ailments has become commonplace. Nonetheless, there is an ever-present challenge to enable less invasive surgical techniques, shorten the time required to surgically implant these devices, decrease patient recovery time, and/or provide other improvements. Thus, there is a need for additional contributions in this area of technology such as presented by the present invention.

### SUMMARY

According to one aspect of the invention an intervertebral body device for implantation within an intervertebral disc space between two adjacent vertebral bodies each having an endplate facing the disc space is disclosed. The device includes two or more bodies interconnected via at least one connecting means such that at least one body is permitted relative motion with respect to the at least one other body. The device has an undeployed configuration and at least one deployed configuration. While in the undeployed configuration, the device has at least one aspect, dimension or area associated with a projection of the device onto a plane. The at least one aspect, dimension or area is increased while in the at least one deployed configuration relative to the undeployed configuration. The device has an axis normal to the projection plane and is configured to be delivered to the intervertebral disc space in the undeployed configuration and subsequently arranged into the at least one deployed configuration for residence in the intervertebral disc space. The deployed configuration comprises one of the bodies being displaced with respect to at least one other body laterally from the axis within the intervertebral disc space.

According to another aspect of the invention an intervertebral body device for implantation within an intervertebral disc space between two adjacent vertebral bodies is disclosed. The device includes two or more bodies interconnected end to end via at least one connecting means such that each body is configured for relative motion with respect to the at least one other adjacent body such that the device is configured to assume various configurations including a first configuration suitable for minimally invasive insertion and a second configuration suitable for residence within the disc space. In the second configuration, at least one of the two or more bodies is displaced relative to another body such that when resident in the intervertebral space the device provides greater stabilizing support to the vertebral bodies between which the device is placed relative to the stabilizing support provided by the device while in the first configuration.

According to another aspect of the invention a system for a disc space between two intervertebral bodies is disclosed. The system includes a device and a device insertion instrument. The device comprises two or more bodies interconnected via at least one connecting means such that at least one body is permitted relative motion with respect to the at least one other body such that the device is configured to assume various configurations including a first configuration suitable for minimally invasive insertion and a second configuration suitable for residence within the disc space. While in the second configuration, at least one of the two or more bodies is displaced relative to another body. The insertion instrument includes a device receiving portion configured to receive the device in the first configuration for delivery to the intervertebral disc space. The device has at least one aspect or dimension associated with a projection of the device onto a plane that is larger in the second configuration relative to the first configuration. The at least one aspect or dimension is in a transverse plane between the two vertebral bodies.

Other advantages will be apparent from the description that follows, including the drawings and claims.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. All figures herein illustrate surgical retractors according to the present invention.

FIG. 1 illustrates a top view of an interbody device disposed within an insertion instrument according to the present invention.

FIG. 2 illustrates a top view of an interbody device partially disposed within an insertion instrument according to the present invention.

FIG. 3 illustrates a top view of an interbody device according to the present invention.

FIG. 4 illustrates a top view of an interbody device in a deployed configuration according to the present invention.

FIG. 5 illustrates a side view of an interbody device disposed inside an insertion instrument located between two vertebral bodies according to the present invention.

FIG. 6a illustrates a perspective view of an interbody device in an undeployed configuration according to the present invention.

FIG. 6b illustrates a perspective view of an interbody device in a deployed configuration according to the present invention.

FIG. 6c illustrates a perspective view of an interbody device in a deployed configuration according to the present invention.

FIG. 7 illustrates a top view of an interbody device according to the present invention positioned relative to a vertebral body.

FIG. 8 illustrates a top view of an interbody device according to the present invention.

FIG. 9 illustrates a side view of the interbody device of FIG. 8 according to the present invention.

FIG. 10 illustrates a perspective view of an interbody device in an undeployed configuration according to the present invention.

FIG. 11 illustrates a perspective view of the interbody device of FIG. 10 in a deployed configuration according to the present invention.

FIG. 12 illustrates a top view of an interbody device according to the present invention.

FIG. 13 illustrates a top view of an interbody device and an insertion instrument according to the present invention.

FIG. 14 illustrates a top view of an interbody device according to the present invention.

FIG. 15 illustrates a side view of an interbody device according to the present invention.

FIG. 16 illustrates a top view of a segment of an interbody device according to the present invention.

FIG. 17 illustrates a top view of an interbody device with shape control means and direction control means according to the present invention.

FIG. 18 illustrates a top view of an interbody device according to the present invention.

FIG. 19 illustrates a top view of an interbody device according to the present invention.

FIG. 20 illustrates a top view of an interbody device according to the present invention.

FIG. 21 illustrates a top view of an interbody device according to the present invention positioned relative to a vertebral body.

FIG. 22 illustrates a top view of an interbody device in a deployed configuration according to the present invention.

FIG. 23 illustrates a top view of a second mechanism of an insertion instrument according to the present invention.

FIG. 24 illustrates a top view of an interbody device and insertion instrument according to the present invention.

FIG. 25 illustrates a top view of an interbody device in an undeployed configuration according to the present invention.

FIG. 26 illustrates a top view of an interbody device in a deployed configuration according to the present invention.

### DETAILED DESCRIPTION

Before the subject devices, systems and methods are described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a spinal segment” may include a plurality of such spinal segments and reference to “the screw” includes reference to one or more screws and equivalents thereof known to those skilled in the art, and so forth.

All publications mentioned herein are incorporated herein by reference to disclose and describe the methods and/or materials in connection with which the publications are cited. Nothing herein is to be construed as an admission that the present invention is not entitled to antedate such publication by virtue of prior invention. Further, the dates of publication provided may be different from the actual publication dates which may need to be independently confirmed.

The present invention is described in the accompanying figures and text as understood by a person having ordinary skill in the field of spinal implants.

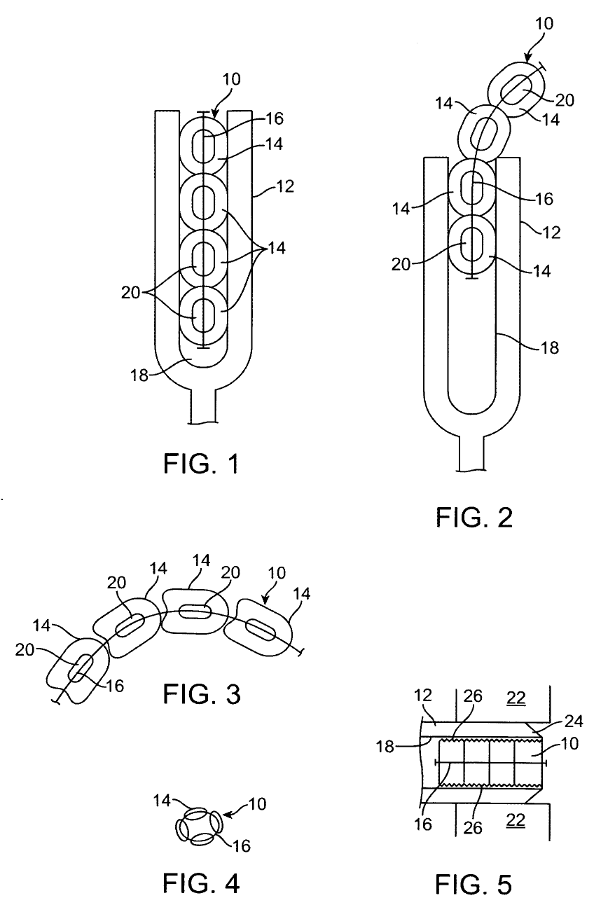

Referring now to FIGS. 1-5, there is shown an interbody device 10 for implantation within an intervertebral disc space according to the present invention. The device 10 comprises more than one body 14 interconnected together with connecting means 16 such that the bodies 14 are capable of relative motion with respect to one another. Each body 14 is shown to include a graft window 20 for placement of bone graft inside the window and subsequent implantation into the intervertebral disc space. FIGS. 1-5 show a bone graft window 20 in each body 14; however, the invention is not so limited and none of the bodies or one or more of the bodies 14 may include one or more bone graft window 20. The bodies 14 also include one or more textured surfaces 26 for traction with the endplates of adjacent vertebral bodies as shown in FIG. 5.

The connecting means 16 shown in FIGS. 1-5 is a cable or wire that connects all of the bodies 14 together; however, the invention is not so limited and any connecting means functionally connecting two or more bodies such that at least one of the bodies is movable relative to at least one other body is within the spirit and scope of the present invention. In one variation, at least a portion of the cable or wire is pretensioned and in another it is not. Furthermore, although a single connecting means 16 is employed in the figures, the device 10 may employ more than one connecting means 16 for connecting two or more of the bodies 14 together.

In FIG. 1, the device 10 is shown disposed inside a receiving portion 18 of an insertion instrument 12 and in a substantially linear or straightened configuration. With the device 10 inside the instrument 12, the device 10 is ready for delivery and placement in an intervertebral disc space. Because the bodies 14 are configured for relative motion with respect to one another, the device 10 is capable of assuming more than one configuration. This feature is particularly advantageous where one configuration is an undeployed configuration suitable for delivery and another configuration is a deployed configuration suitable for residence within the intervertebral disc space. Even greater advantage is provided by the device 10 of the present invention when, in the undeployed configuration, an aspect or dimension of the device 10 is smaller than the same aspect or dimension while in the deployed configuration. This feature enables the device 10 to be implanted using a surgical procedure that is less invasive than if the device 10 was entirely rigid, that is, without body components 14 that are capable of relative motion with respect to one another such that an aspect or dimension of the device can be reduced. Of course, minimally invasive implantation is accomplished by orienting the device along its relatively smaller aspect or dimension for insertion through an incision or opening in the patient as small as necessary to insert the device along this smaller aspect or dimension. Another advantage of the device 10 is that the deployed configuration can be customized according to surgeon preference and/or patient anatomy. These and other advantages will become more evident from the figures and related discussion hereinbelow.

Referring now to FIG. 2, there is shown a device 10 according to the present invention partially disposed inside the insertion instrument 12. Partial insertion or removal of the device in this variation frees the bodies that are outside of the insertion instrument for movement relative to one another. This relative motion of the bodies with respect to one another can be automatically or manually induced. In one variation, the relative movement is caused by the connecting means being a pre-tensioned cable or wire forcing the bodies into a desired configuration such as the configuration shown in FIG. 3 which illustrates a device according to the present invention in a deployed configuration completely retracted from the insertion instrument or prior to insertion into the insertion instrument. The deployed configuration shown in FIG. 3 is slightly curved to conform to the natural curvature of the vertebral body or of a shape typical of interbody spinal devices. Another deployed configuration is shown in FIG. 4 which is a generally circular configuration. Any deployed configuration is possible and is only limited by the number and geometry of the bodies as well as the type of the connecting means that is employed. The deployed configuration involves one of the bodies being moved relative to at least one other body laterally or in a transverse plane between the adjacent vertebral bodies.

With particular reference now to FIGS. 1, 2 and 5, the insertion instrument 12 generally includes a device receiving portion 18 configured to receive the device 10. The receiving portion 18 is shown to have a substantially rectangular shape, however the invention is not so limited and the receiving portion can have any geometry so long as it is complementary to the geometry of the device in at least one configuration. The geometry of the receiving portion 18 of the insertion instrument 12 defines the undeployed configuration of the device and forces the device 10 into such undeployed configuration as it is inserted therein wherein the opening of the receiving portion is dimensioned to accept the smaller aspect or dimension of the device for minimally invasive insertion into the patient.

With particular reference to FIG. 5, the insertion instrument 12 is sized slightly larger than the distance between the endplates of adjacent vertebral bodies 22 to assist in distracting the adjacent bodies and/or to give the device 10 freedom of motion to assume its deployed configuration upon removal from the insertion instrument. In another variation, the insertion instrument includes a leading portion, distractor, expander or guide 24 that is configured to spread apart the adjacent vertebral bodies. The distractor 24 includes angled surfaces, such as in the variation shown in FIG. 5, forming a ramp to distract the vertebral bodies.

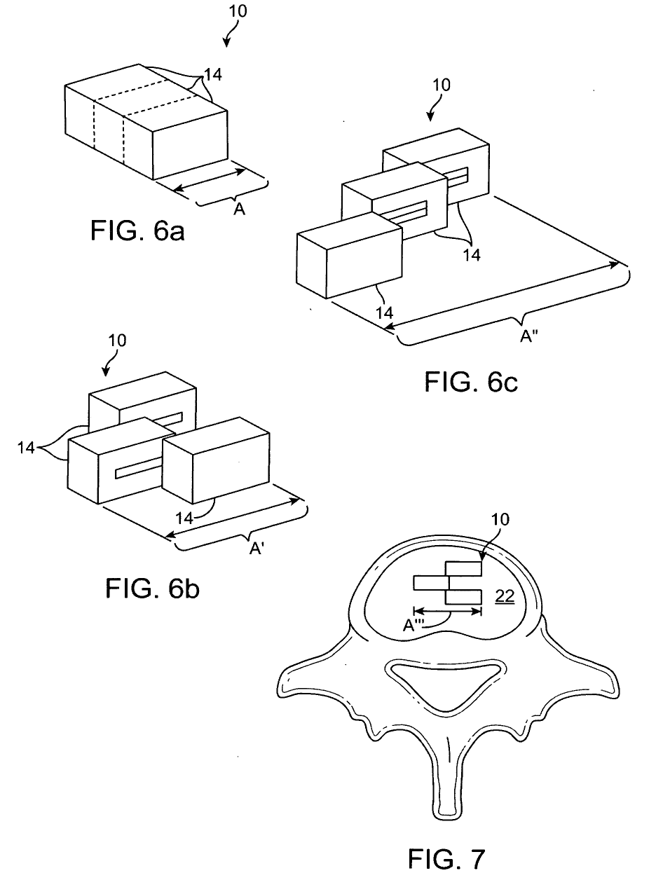

Referring now to FIGS. 6a, 6b, 6c and 7, there is shown another variation of the device 10 according to the present invention. In this variation, the more than one body 14 is connected to an adjacent body 14 along substantially planar surfaces such that the bodies 14 slide relative to one another at the at least one interface. Although planar surfaces are shown, the invention is not limited to planar interfaces so long as the complementary interfaces permit sliding engagement of one body with respect to another body. FIG. 6a shows three bodies 14, each having a substantially rectangular geometry, interconnected at planar interfaces for sliding engagement. Although three bodies are shown and their geometry is substantially rectangular, the invention is not so limited and any shape and number of bodies are possible. The connecting means 16 is such that sliding engagement of device bodies 14 relative to one another is permitted. In FIGS. 6 and 7, the connecting means 16 is at least one pin and slot.

Still referencing FIGS. 6a, 6b, 6c and 7, the device 10 in FIG. 6a is shown in an undeployed configuration whereas the devices in FIGS. 6b, 6c and 7 are shown in various deployed configurations. One aspect or dimension A of the undeployed configuration of FIG. 6a is smaller than the same aspect or dimension A¹, A¹¹ and A¹¹¹ of deployed configurations shown in FIG. 6b, FIG. 6c and FIG. 7, respectively. Because the aspect A is smaller in the undeployed configuration, the device is suitably adapted for less invasive implantation while affording the structural stability of an otherwise non-dynamic or non-expandable device when in the deployed configuration. The one aspect or dimension A lies in a projection plane of the device wherein the projection of the one aspect or dimension of the device is preferably smaller in an undeployed configuration relative to a deployed configuration. Less invasive implantation is accomplished along an axis of the device that is substantially perpendicular to a projection plane of the device having the projection area or one dimension or aspect in the projection plane that is smaller in the undeployed configuration relative to another projection of the device. The device is oriented along this axis and delivered to the operative site. The multi-unit device 10 expands laterally from the configuration of FIG. 6a to the configurations shown in FIGS. 6b, 6c and 7 to increase vertebral body coverage along at least one aspect or projection area or projection dimension in the plane parallel to a vertebral body endplate. FIG. 7 is a top view of the device shown disposed relative to a vertebral body.

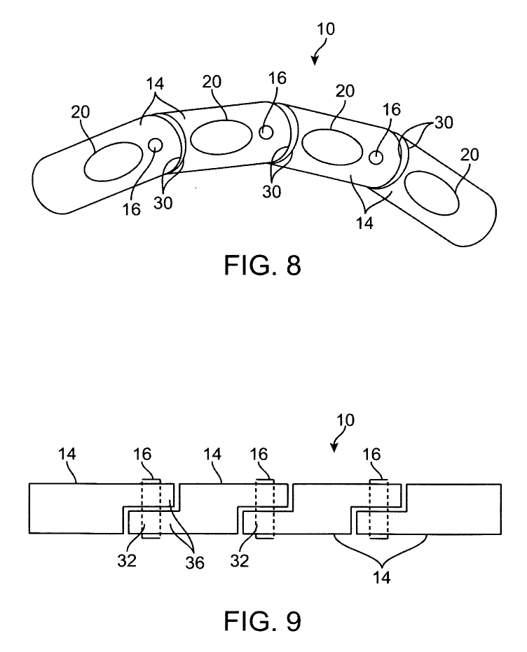

Referring now to FIGS. 8 and 9, there is shown another interbody device 10 according to the present invention. FIG. 8 is a top view of the device 10 showing graft windows 20 and bodies 14 having non-planar, complementary engaging surfaces 30 that permit movement of one body 14 relative to an adjacent body 14. In FIG. 8, the device 10 is shown having a slight curvature and may be considered to be in a deployed or partially deployed configuration. In FIG. 9 which is a side-elevational view, the device 10 is shown in a straightened or undeployed configuration. Motion of bodies 14 with respect to one another is permitted by connecting means 16. In this variation, the connecting means 16 is a pin and hole configuration such that a pin 32 passes through holes formed in adjacent bodies 14 to link adjacent bodies together and permit relative rotation of one or two bodies about the pin 32. A plurality of the same connecting means is employed to connect the plurality of bodies. In another variation, the same connecting means is not employed throughout but different connecting means are employed in the same device. The bodies 14 have complementary geometries that permits one pin 32 to pass through two adjacent bodies. In the variation shown in FIG. 9, the geometry of the bodies is such that the bodies include overlapping extending flange portions 36 with the pin holes 34 being located in the flange portions 36. Similarly as described above, at least one aspect of the device 10 in the undeployed configuration is smaller than in the deployed configuration permitting a more minimally invasive deployment into the patient.

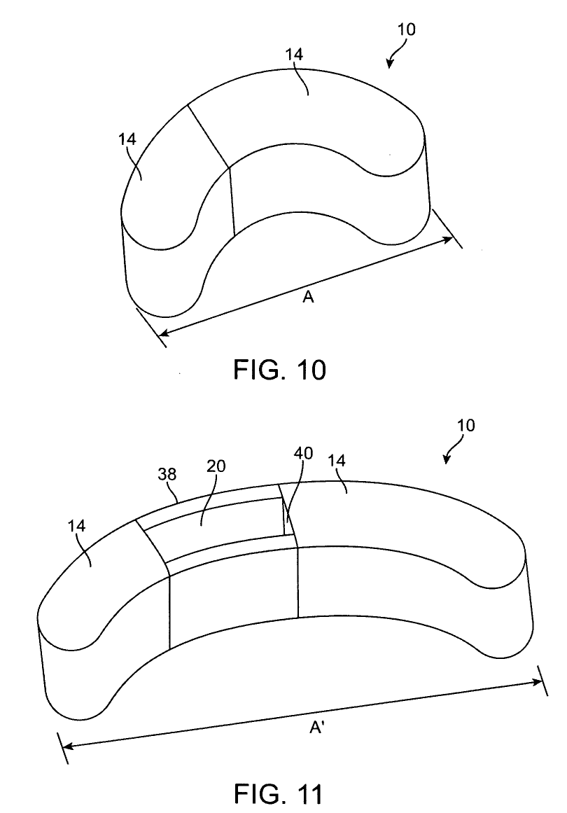

Referring now to FIG. 10 and 11, there is shown another variation of the interbody device 10 according to the present invention. The device 10 in FIG. 10 is shown in an undeployed configuration and in a deployed configuration in FIG. 11. The device 10 includes at least two body portions 14 adapted for sliding engagement relative to one another. One of the bodies 14 includes an extending portion 38 configured to be receiving inside a receiving portion 40 of an adjacent body 14. The receiving portion 40 and extending portion 38 have complementary geometries wherein the extending portion 38 is sized slightly smaller than the receiving portion 40. The extending portion 38 and receiving portion 40 comprise the connecting means for this variation. Stops can be formed to prevent the extending portion 38 from completely sliding out of the receiving portion 40. The extending portion 38 advantageously includes a graft window 20 that opens when the receiving portion 40 of the adjacent body 14 slides into the deployed configuration. Although one window is shown, alternative variations with more than one graft window 20 are within the scope of the present invention. Again, at least one aspect or dimension A of the undeployed configuration is smaller than the same aspect or dimension A¹ of the deployed configuration which advantageously permits minimally invasive deployment. The device 10 extends laterally increasing vertebral body end plate projection coverage. Also, even though the device 10 is shown having a curved shape, the invention is not so limited any the device can be any shape such as elliptical, circular, polygonal, square, rectangular, etc.

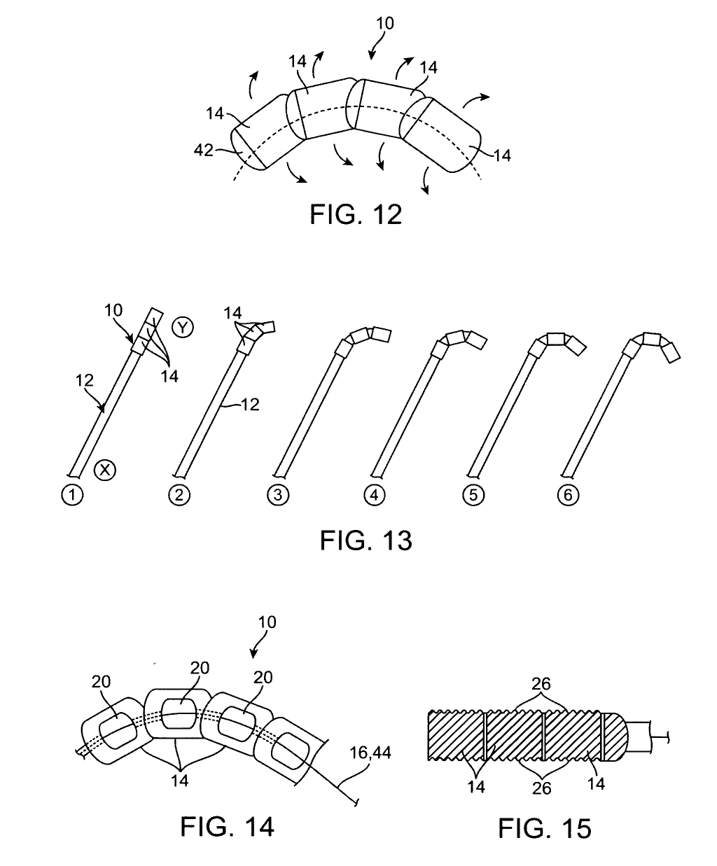

Referring now to FIGS. 12-18, there is shown another variation of the mutli-component interbody spacer 10 according to the present invention. The device 10 includes more than one body 14 interconnected together with connecting means such that the bodies are capable of relative motion with respect to one another as shown by the arrows in FIG. 12. In one variation, an end body of the device 10 includes a distractor or guide 42 having a curved or ramped surface connected to or integrally formed with the end-body 14 or as a separate body which assists in the insertion of the device 10. In one variation, the snake-like device 10 includes bone graft windows 20 as shown in FIG. 14. Also, one or more textured surface 26 is provided for traction with the endplates of the vertebral bodies as shown in FIG. 15.

FIG. 13 shows the device 10 connected to a distal end of an insertion instrument 12. A stepwise progression of motion of the bodies 14 relative to one another from an undeployed to a deployed configuration is shown in FIG. 13 as controlled at the proximal end x of the insertion instrument 12. The configuration of the device 10 is controlled via control means 44. In one variation, the control means 44 is a cable, wire, cord or band that can be made of metal, plastic, memory shape material or any other suitable material. The control means 44 extends through the bodies 14 of the device 10 and also extends through the length of the insertion instrument 12 to a proximally positioned controller (not shown) that controls and locks the device configuration into place. In one variation in which the control means 44 is a cable or wire, the controller adjusts the cable tension at the proximal end of the insertion instrument 12 to effect a change in configuration of the device 10 at the distal end as shown in FIG. 13. In another variation, the controller pulls and releases the wire to to effect a change in configuration of the device. In one variation, the control means 44 comprises the connecting means 16. In another variation, the connecting means 16 comprises the control means 44. In another variation, the control means 44 is independent of the connecting means 16 and vice versa; and, yet in another variation, the control means 44 and connecting means 16 are interdependent such that (1) the connecting means 16 plays a role, via camming surfaces and the like, in controlling the outcome of the final configuration and/or (2) the control means 44 plays a role in connecting the bodies together.

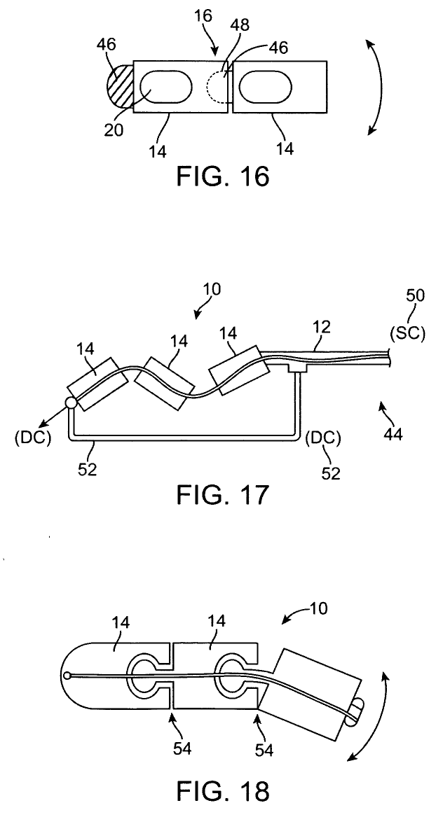

Referring now to FIG. 16, there is shown a segment of the interbody device 10 according to the present invention wherein the connecting means 16 is a ball and socket type configuration of the bodies. One or more bodies include a ball 46 configured to be at least partially received and retained inside the socket 48 of an adjacent body 14. In one variation, the ball and socket connecting means 16 is advantageously configured to permit polyaxial rotation of one body with respect to an adjacent body and adjustment permitting the device to snake up and down as well as sideways and, thereby, snake through and adapt to complex patient anatomy or irregular vertebral body end plate surfaces.

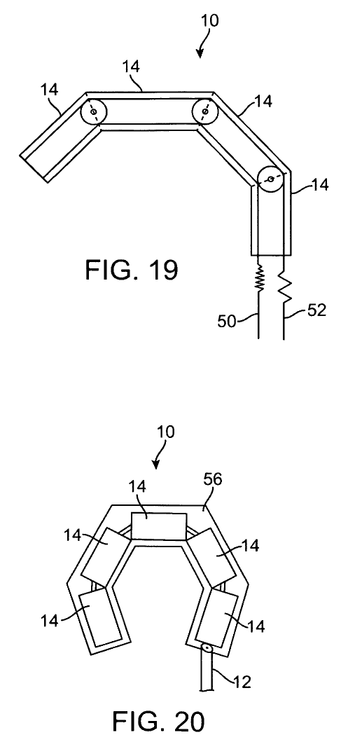

Referring now to FIG. 17, there is shown a device 10 connected to an insertion instrument 12 according to the present invention. In the variation shown in FIG. 17, the control means 44 is bifurcated into shape control means 50 and direction control means 52. Shape control means 50 can be any means for controlling the configuration of the bodies 14 and direction control means 52 can be any means for controlling the direction of the device 10. In one variation, both the shape control means 50 and direction control means 52 are connected to the same controller (not shown) at the proximal end of the insertion instrument 12. In another variation, the shape control means 50 and the direction control means 52 are connected to separate controllers at the same or different location as illustrated in FIG. 19. As shown in FIG. 17, the direction control means 52 is connected to the distal end of the device 10 and is configured to lead the device by the distal end to the desired implantation location such as the inter-vertebral body disc space. Once in position, the shape control means 50 is activated to effect a desired deployed configuration of the device. In one variation, the bodies 14 of the device have specialized interfacing abutments 54 configured to define the deployed configuration as shown in FIG. 18. Once in the final position, the configuration of the device 10 is fixed with suitable means such as a crimp, screw, nut, wedge, lock or any other suitable means within the scope and spirit of the present invention.

Referring now to FIG. 19, there is shown an interbody device 10 having interconnected bodies 14 according to the present invention. FIG. 19 illustrates shape control means 50 and direction control means 52. In one variation, both the shape control means 50 and direction control means 52 are connected to the same controller (not shown). In another variation, the shape control means 50 and the direction control means 52 are connected to separate controllers at the same or different locations. In FIG. 19, two guides, wires and/or cables with variable tension adjustment are employed as shape control means and direction control means. The two can be positioned substantially parallel to each other or not. Furthermore, the one or more of the shape control means and direction control means 50, 52 are located inside the device 10 as shown in FIG. 19 or can be located outside of the device 10. Also, the means 50, 52 are centered in the device in another variation.

Referring now to FIG. 20, there is shown an interbody device 10 having interconnected bodies 14 according to the present invention. The device 10 of FIG. 20 includes an outer cover 56 configured to encase one or more of the connecting means 16, control means 44, shape control means 50 and direction control means 52. In one variation, the encasement 56 is removable after deployment along with one or more of the connecting means 16, control means 44, shape control means 50 and direction control means 52.

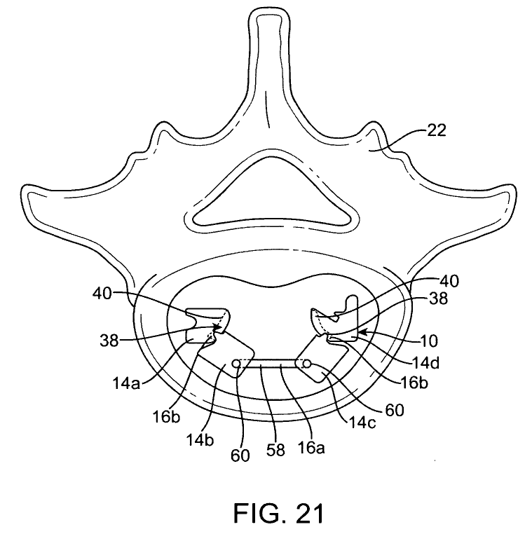

Referring now to FIGS. 21-26, and in particular, to FIG. 21, there is shown another variation of a multi-body intervertebral body device 10 positioned relative to a vertebral body 22 according to the present invention. The device 10 includes four interconnected bodies 14a, 14b, 14c and 14d, but the invention is not so limited and any number of bodies 14 functionally arranged is within the scope of the present invention. The bodies 14 are connected via at least one type of connecting means 16 such that relative motion with respect to adjacent bodies is permitted.

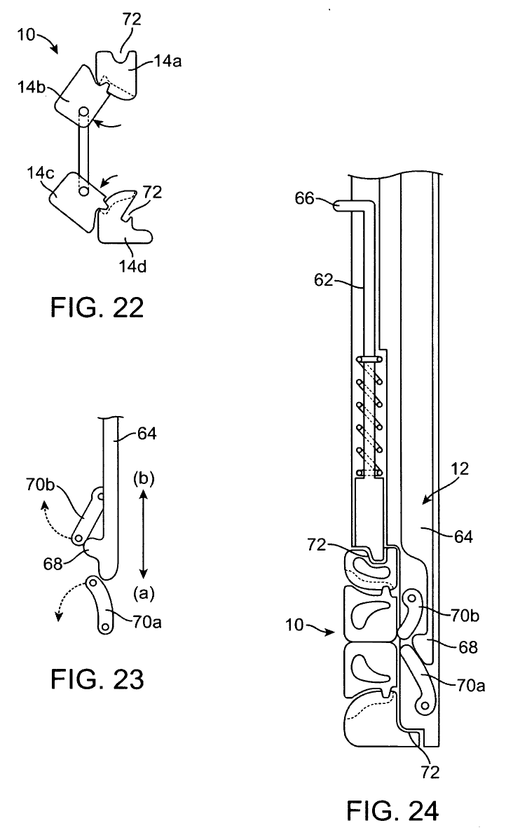

Still referencing FIG. 21, the device 10 includes two types of connecting means 16a and 16b. The first connecting means 16a comprises a rod 58 connected to bodies 14b and 14c such that bodies 14b, 14c are configured for rotation about the rod-to-body connection points 60. In one variation, the bodies 14b and 14c are connected via rod 58 such that the bodies 14b, 14c are capable of translation with respect to the rod 58 in addition to rotation with respect to the rod 58. In such a variation, the rod 58 is attached to bodies 14b and 14c via a slotted pinned connection. In another variation, the bodies 14b and 14c are capable of translation without rotation with respect to the rod 58. Body 14a is connected to body 14b via a second connecting means 16b as is body 14d and 14c wherein the first and second connecting means 16a, 16b are different. In another variation the first and second connecting means 16a, 16b are the same. Second connecting means 16b include an extending portion 38 formed on one body 14b and a receiving portion 40 formed on an adjacent body such as body 14a. The extending portion 38 is configured to be received inside the receiving portion 40 and in one variation, the receiving portion 40 is an elongated slot such that the extending portion 38 is allowed to slide along inside the receiving portion 40. Also, the bodies 14 include conforming complementary surfaces for ease of translation and rotation of the bodies from an undeployed configuration to a deployed configuration and to help define the resultant configurations. Also, the end bodies 14a and 14d additionally include instrument engagement features 72 as shown in FIG. 22 configured to engage the instrument 12 as shown in FIG. 24 so that it is retained inside the insertion instrument 12.

With reference now to FIG. 24, there is shown the device 10 of FIG. 21 in an undeployed configuration connected to an insertion instrument 12. The insertion instrument 12 includes a first mechanism 62 and a second mechanism 64. The first mechanism 62 is connected to the device 10 in a spring biased fashion to keep it connected to the insertion instrument 12. The first mechanism 62 is pulled back via the trigger 66 to release the device 10 from the instrument 12. The second mechanism 64 includes at least one caming surface 68 at the distal end of the mechanism 62 and two hinged levers 70a, 70b that are configured to engage the at least one caming surface 68. As shown in FIG. 23, when the second mechanism 64 is pushed toward the distal end of the instrument 12 in direction (a), the at least one camming surface 68 forces lever 70a to pivot about its pinned connection to the instrument 12, contact the device 10 and advance the device 10 outwardly as shown in FIG. 22. The insertion instrument 12 advantageously includes a side deployment window. When the second mechanism 64 is retracted in a proximal direction (b), the at least one caming surface 68 contacts lever 70b and forces lever 70b to pivot about its pinned connection to the instrument 12, contact the device and advance the device outwardly as shown in FIG. 22.

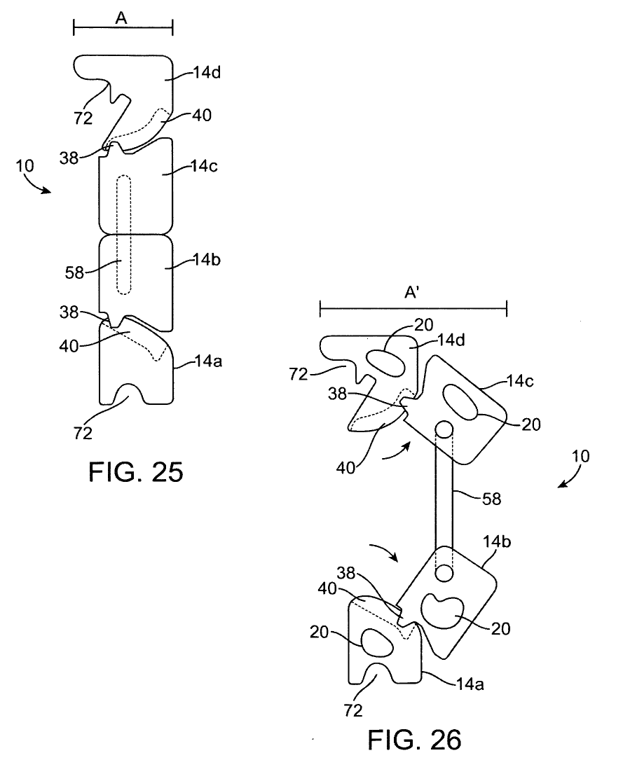

Referring now to FIGS. 25 and 26, the device 10 of FIG. 21 is shown in an undeployed configuration in FIG. 25 such that at least one aspect or dimension A is smaller than the same aspect or dimension A¹ when in a deployed configuration shown in FIG. 26. In one variation, dimension A is approximately between 0.2 inches and 0.5 inches and dimension A¹ is approximately between 0.5 inches and 1.0 inches. FIGS. 25 and 26 show the lateral translation of bodies 14c and 14d as well as their rotation with respect to the rod 58. FIGS. 25 and 26 also show the relative movement of end bodies 14d and 14a.

As seen in FIG. 26, the device 10 in the deployed configuration advantageously leaves a lot of space for graft material to be disposed around the device 10 as well as inside graft windows 20. The device bodies 14 are typically made of any suitable biocompatible material such as titanium, surgical steel and PEEK and the rod 58 is made from the same or other suitable material. Of course, a cannula may be employed to insert and deliver any of the devices described above. The device of FIGS. 21-26 is particularly adapted for lateral interbody fusion, providing minimal morbidity while allowing placement of large interbody implants and grafts and restoration of disc height. Furthermore, the device is suitable with complete discectomy providing restoration of disc height with indirect foraminal and canal decompression. Overall, the multi-body intervertebral body device of the present invention provides for a minimally-invasive approach with maximal access and can be employed with a retroperitoneal, transpsoas approach to the lumbar spine for treatment of lumbar degenerative disorders.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

We claim:

1. An intervertebral body device for implantation within an intervertebral disc space between two adjacent vertebral bodies each having an endplate facing the disc space, comprising:
two or more bodies interconnected via at least one connecting means such that at least one body is permitted relative motion with respect to the at least one other body;
wherein the device has an undeployed configuration and at least one deployed configuration;
wherein the device, while in the undeployed configuration, has at least one aspect, dimension or area associated with a projection of the device onto a plane; the device having an axis normal to said plane;
wherein the at least one aspect, dimension or area is increased while in the at least one deployed configuration relative to the undeployed configuration; and
wherein the device is configured to be delivered to the intervertebral disc space in the undeployed configuration and subsequently arranged into the at least one deployed configuration for residence in the intervertebral disc space; said deployed configuration comprising one of said bodies displaced with respect to at least one other body laterally from the axis within the intervertebral disc space.

2. The device of claim 1 wherein the relative motion of at least one body with respect to at least one other body is a sliding motion.

3. The device of claim 1 wherein the relative motion of at least one body with respect to at least one other body is a polyaxial motion.

4. The device of claim 1 wherein at least one body includes a graft window.

5. The device of claim 1 wherein adjacent bodies define an interface therebetween.

6. The device of claim 5 wherein the interface is of a type selected from a group consisting of a planar interface, a complimentary interface, and a curved interface.

7. The device of claim 1 wherein one body includes an extending portion and an adjacent body includes a receiving portion wherein the receiving portion and the extending portion have complimentary geometries and are configured such that the extending portion is received at least partially within the receiving portion and configured for relative motion with respect to one another such that the extending portion is movable inside the receiving portion to and from the undeployed configuration and a deployed configuration.

8. The device of claim 1 wherein the device includes an end body configured to distract the adjacent vertebral bodies.

9. The device of claim 1 including a first body connected to a second body via a first connecting means, the second body connected to a third body via a second connecting means, the third body connected to a fourth body via a third connecting means.

10. The device of claim 9 wherein the first and third connecting means are the same; and the first and third connecting means are different from the second connecting means.

11. The device of claim 9 wherein the second connecting means is a rod with the second and third bodies being slidable and rotatable with respect to said rod.

12. The device of claim 11 wherein the first and third connecting means is a slot-pin connection configured such that the first body is capable of relative motion with respect to the second body and the fourth body is capable of relative motion with respect to the third body.

11. The device of claim 1 wherein the two or more bodies are sequentially connected with said at least one connecting means.

12. The device of claim 1 wherein the connecting means is selecting from a group consisting of a cable, wire, pretensioned cable, pretensioned wire, pin-and-slot, pin-and-hole, extending portion and receiving portion, and ball and socket.

13. The device of claim 1 wherein the two or more bodies are rectangular bodies.

14. The device of claim 1 wherein the deployed configuration is selected from a group consisting of curved, banana-shaped, and circular.

15. The device of claim 1 wherein the undeployed configuration is configured for minimal invasive insertion into the patient.

16. The device of claim 1 wherein the device is configured for deployment through a cannula.

17. The device of claim 1 wherein the connecting means extends through and connects all of the bodies.

18. The device of claim 1 further including control means.

19. The device of claim 18 wherein the control means is selected from a group consisting of a cable, wire, cord and band.

20. The device of claim 18 further including a controller connected to the proximal end of the control means configured to control the control means.

21. The device of claim 18 further including a controller connected to the control means; the controller being configured to manipulate the control means to effect change in configuration of the device.

22. The device of claim 18 wherein the control means includes a wire; the device further including a controller configured to adjust the tension of the wire to change the configuration of the device.

23. The device of claim 18 wherein the control means includes:
a shape control means to effect the desired deployed configuration, and
a direction control means configured to lead the device to the desired implantation location.

24. An intervertebral body device for implantation within an intervertebral disc space between two adjacent vertebral bodies, comprising:
two or more bodies interconnected end to end via at least one connecting means such that each body is configured for relative motion with respect to the at least one other adjacent body such that the device is configured to assume various configurations including a first configuration suitable for minimally invasive insertion and a second configuration suitable for residence within the disc space wherein at least one of the two or more bodies is displaced relative to another body in the second configuration such that when resident in the intervertebral space the device provides greater stabilizing support to the vertebral bodies between which it is placed relative to the stabilizing support provided by the device while in the first configuration.

25. The device of claim 24 wherein a single connecting means interconnects all of the two or more bodies.

26. The device of claim 24 wherein a connecting means interconnects adjacent bodies.

27. The device of claim 24 wherein the connecting means imparts the device with snake-like characteristics.

28. The device of claim 24 further including shape control means.

29. The device of claim 28 wherein the connecting means is a wire interconnecting all of the bodies and the shape control means pulls or releases the wire to effect a change in configuration of the device.

30. The device of claim 24 further including direction control means configured to lead the device into position.

31. A system for a disc space between two intervertebral bodies comprising:
a device comprising two or more bodies interconnected via at least one connecting means such that at least one body is permitted relative motion with respect to the at least one other body such that the device is configured to assume various configurations including a first configuration suitable for minimally invasive insertion and a second configuration suitable for residence within the disc space wherein at least one of the two or more bodies is displaced relative to another body while in the second configuration;
an insertion instrument comprising a device receiving portion configured to receive the device in the first configuration for delivery to the intervertebral disc space; and
wherein the device has at least one aspect, dimension or area associated with a projection of the device onto a plane that is larger in the second configuration relative to the first configuration; said at least one aspect or dimension lying in a transverse plane between the two vertebral bodies.

32. The system of claim 31 further including a first mechanism configured to retain the device inside the device receiving portion and a second mechanism configured to deploy the device from the insertion instrument.

33. The system of claim 32 wherein the second mechanism includes one caming surface configured to engage a first hinged lever and a second hinged lever; each of said levers activable to emit at least one portion of the device from the insertion instrument.
